(19) United States
(12) Patent Application Publication
(10) Pub. No.: US 2012/0065535 A1
(43) Pub. Date: Mar. 15, 2012

(54) GAS ANALYSIS APPARATUS HAVING A COMBINATION OF GAS DEHUMIDIFIER AND GAS CONVERTER

(76) Inventors: Klaus Abraham-Fuchs, Erlangen (DE); Maximilian Fleischer, Hohenkirchen (DE); Karsten Hiltawsky, Schwerte (DE); Oliver Hornung, Furth (DE); Thomas Krüger-Sundhaus, Pommersfelden (DE); Erhard Magori, Feldkirchen (DE); Peter Paulicka, Erlangen (DE); Oliver Von Sicard, Munchen (DE)

(21) Appl. No.: 13/263,873

(22) PCT Filed: Mar. 19, 2010
(86) PCT No.: PCT/EP2010/053590

§ 371 Date: Oct. 11, 2011

(30) Foreign Application Priority Data
Apr. 8, 2009 (DE) 10 2009 016 848.6

(51) Int. Cl. A61B 5/097

(52) U.S. Cl. 600/532

(57) ABSTRACT

An apparatus for measuring nitrogen monoxide in exhaled air by way of a gas sensor unit (13) having at least one gas sensor (15), has a device for gas conversion (11), wherein a device for gas dehumidification (19) is connected upstream of the device for gas conversion.

### CROSS-REFERENCE TO RELATED APPLICATIONS

This application is a U.S. National Stage Application of International Application No. PCT/EP2010/053590 filed Mar. 19, 2010, which designates the United States of America, and claims priority to German Application No. 10 2009 016 848.6 filed Apr. 8, 2009. The contents of which are hereby incorporated by reference in their entirety.

### TECHNICAL FIELD

The present invention relates to an arrangement for measuring the concentration of nitric oxide (NO) in respiratory gas and to a method for measuring the concentration of NO.

### BACKGROUND

Nitric oxide (nitrogen monoxide, NO) is continuously released into the respiratory-gas flow in very small quantities from the cells of the respiratory tract. NO constitutes an important marker for the diagnosis and optimized therapy of asthma and other inflammatory respiratory diseases. With a prevalence of approximately 5% in adults and approximately 20% in children, asthma is one of the most common diseases in developed industrialized countries. In inflammatory processes in the respiratory tract, e.g. asthma, there are increased NO concentrations of 80 ppb (parts per billion) in the exhaled air. Imminent asthma attacks can be identified significantly earlier as a result of an increase of the NO content in the exhaled air than as a result of a pulmonary function test. Hence the NO measurement in the exhaled air is a preferred method for diagnosing and monitoring the therapy of asthma and other inflammatory respiratory diseases.

Cost-effective NO sensors with the required sensitivity in the ppb-range have not been commercially available until now. A newly developed NO₂-sensor on the basis of the “suspended gate FET” technology meets the aforementioned demands. However, a conversion module for converting NO in the respiratory gas into NO₂, which can be detected by the sensor, must be placed upstream of such a sensor. Ideally, such a conversion module should last a number of months or even years, be cost-effective and convert NO into NO₂ with the highest possible and constant conversion rate. Nitrogen monoxide is converted into nitrogen dioxide according to the following reaction equation:

2 NO+O₂ [symbol] 2 NO₂.

Nitrogen monoxide can be converted to nitrogen dioxide in a respiratory gas sensor instrument by means of a device for oxidizing nitrogen monoxide into nitrogen dioxide, for example by routing the (respiratory) air through an oxidation means (e.g. potassium permanganate) or an oxidation catalyst.

The fact that NO₂ is significantly more soluble in water than NO is a further problem.

### SUMMARY

According to various embodiments, a method is required to keep the concentration of the converted NO₂ in the humid respiratory gas as constant as possible and to keep it quantitatively measurable. As a result of the higher solubility of NO₂ in water, part of the (converted) NO₂ is dissolved in water in (respiratory) air with a high moisture content, the concentration of the measurable NO₂ falls and an apparently too low NO content is measured.

In order to allow a quantitative measurement of the NO content, a respiratory gas analysis instrument is proposed according to various embodiments, in which the respiratory air is firstly routed through a device for air dehumidification and subsequently through a device for oxidizing nitrogen monoxide to nitrogen dioxide.

According to an embodiment, an apparatus for measuring at least one gas analyte in exhaled air using a gas sensor unit with at least one gas sensor, may comprise a device for gas conversion, and a device for air dehumidification arranged upstream of the device for gas conversion.

According to a further embodiment, the apparatus may further comprise an indicator that displays the degree of water absorption or displays if the water content of the air exceeds a critical threshold after flowing through the device for air dehumidification. According to a further embodiment, the apparatus may further comprise a chemical desiccant for dehumidifying air. According to a further embodiment, the apparatus may further comprise an apparatus for regenerating the chemical desiccant. According to a further embodiment, the apparatus may further comprise an electrical drying means for dehumidifying air. According to a further embodiment, wherein at least one of the device for air dehumidification and the device for gas conversion are provided as a consumable in a separate unit. According to a further embodiment, the apparatus may further comprise a particle filter. According to a further embodiment, the apparatus may further comprise a one-way valve such that a user is no longer able to suck in and re-inhale air already exhaled into the apparatus. According to a further embodiment, the at least one gas analyte can be nitrogen monoxide and the device for gas conversion can be a device for oxidizing nitrogen monoxide to nitrogen dioxide. According to a further embodiment, the gas sensor can be an NO₂-sensitive FET-sensor.

### BRIEF DESCRIPTION OF THE DRAWINGS

The invention will be described below on the basis of examples and in conjunction with the figure, in which:

FIG. 1 shows a schematic illustration of an embodiment of the apparatus.

### DETAILED DESCRIPTION

In addition to stabilizing and reducing the amount of NO₂ that dissolves in the air moisture, this method is additionally advantageous in that the dehumidification of the air reduces a possibly present cross sensitivity of the gas sensor to air moisture, or brings said cross sensitivity to a defined value as a result of the defined setting of the air moisture. Furthermore, this optionally allows instruments with significantly reduced energy requirements: respiratory gas with a dew point of typically 35-38° C. would, in an instrument at room temperature, cause condensation of the moisture which would, possibly, lead to falsification of measurements or, if the measurement chamber is well sealed with respect to the surroundings, lead to the measurement chamber filling up with water. Conventionally, heating the chamber above the dew temperature of the respiratory moisture is used to prevent this effect; however, this has a typical energy requirement of a plurality of W and leads to a waiting period until the instrument is operational (heating-up phase). As a result of the gas dehumidifier this can be dispensed with, and it is possible to design a small instrument with reduced energy requirements.

According to various embodiments, an apparatus for measuring nitrogen monoxide in exhaled air using a gas sensor unit with at least one gas sensor, may have a device for oxidizing nitrogen monoxide to nitrogen dioxide, wherein a device for air dehumidification is arranged upstream of the device for oxidizing nitrogen monoxide to nitrogen dioxide.

In broad terms, various embodiments relate to an apparatus for measuring a gas analyte in exhaled air using a gas sensor unit with at least one gas sensor, having a device for gas conversion, wherein a device for air dehumidification is arranged upstream of the device for gas conversion.

The gas analyte is preferably nitrogen monoxide and the device for gas conversion is a device for oxidizing nitrogen monoxide to nitrogen dioxide.

The gas sensor is preferably an NO₂-sensitive FET sensor.

The oxidation can be brought about by routing the (respiratory) air through an oxidation means (e.g. potassium permanganate) or an oxidation catalyst.

Provision is preferably made for an indicator that displays the degree of water absorption or displays if the water content of the air exceeds a critical threshold after flowing through the device for air dehumidification.

As per one embodiment, use is made of a chemical desiccant for dehumidifying air.

There are different types of desiccants:
dehydration via a chemical reaction=irreversible
water is bound by formation of water of crystallization
reversibly absorbed in molecular sieves

Suitable desiccants preferably are calcium chloride, copper sulfate, silica or zeolites, blue gel, orange gel and the like. Blue gel and orange gel likewise consist of silica gel, but contain an indicator dye that shows the degree of water absorption.

As per one embodiment, the device for air dehumidification is provided as a consumable in a separate unit, which can be replaced separately when the device is used up, e.g. when the water-absorption capacity has been depleted.

As per one embodiment, the device for air dehumidification is an electrical drying means for air dehumidification, e.g. an electrical heating or condensation means.

As per one embodiment, the device for oxidizing nitrogen monoxide to nitrogen dioxide is provided as a consumable in a separate unit, which can be replaced separately when the device is used up, e.g. when the oxidation capacity has been depleted.

As per one embodiment, the device for air dehumidification is provided together with the device for oxidizing nitrogen monoxide to nitrogen dioxide as a consumable in a separate unit. A separate unit as described above is preferably provided in a replaceable fashion on the respiratory gas analysis instrument without requiring further tools, e.g. by one or more simple plug-in connection(s).

As per one embodiment, the gas sensor unit has an NO₂-sensitive field effect transistor sensor (FET sensor).

Since the reaction enthalpy is negative, the reaction is toward the conversion into NO₂; thus, it must only be made possible by a catalyst. In this respect reference is made to the fact that a large part of the oxygen present in the surrounding air is still present even in the exhaled air because only a small part of the oxygen is used up during respiration.

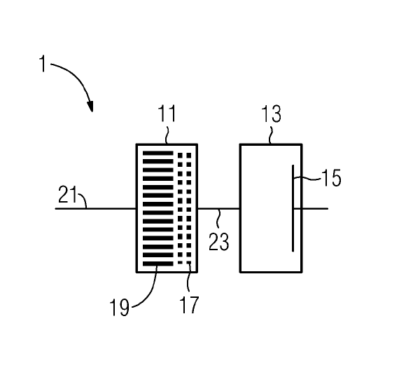

In an exemplary and schematic fashion, FIG. 1 shows an embodiment of the apparatus 1 according to various embodiments with a conversion module 11 and a gas sensor unit 13. Exhaled air is routed via a feed line 21 into a separate unit 11, in which provision is made for a device for oxidizing nitrogen monoxide 17. Arranged upstream thereof is a device for dehumidifying air 19, e.g. in the form of a chemical desiccant such as e.g. silica gel, copper sulfate or the like. After the conversion, the exhaled air is conducted via a line 23 to the gas sensor unit 13, in which provision is made for an NO₂-sensitive gas sensor 15.

Optionally, the separate unit 11, which may be embodied as a disposable, or the flow channel system that is plugged onto the separate unit may contain a one-way valve (e.g. a unidirectional check valve) (not illustrated) such that the user is no longer able to suck in and re-inhale air already exhaled into the module.

The converter and/or the dehumidification module 11 can be embodied such that e.g. a color change or another perceptible change indicates when the module is used up and needs to be replaced. A comparison color scale can be applied next to the window for observing the color change.

Furthermore, the module can optionally contain an apparatus for regenerating the reaction chemicals that are being consumed. Thus, the dehumidification module 11 can contain a heating device, e.g. a heating wire, by means of which the stored moisture can be baked out again. In this case, the module 11 and the analysis instrument have contact interfaces for operating the regeneration apparatus integrated thereon.

In another embodiment, the conversion module and/or the dehumidification module 11 are inserted directly in front of or into the measurement chamber through a duct accessible from the outside such that the converted gas can be measured shortly after the conversion and changes in concentration as a result of the onset of a chemical equilibrium are avoided. In this embodiment, the conversion module and/or the dehumidification module 11 is advantageously situated behind a valve (not shown) in the flow channel system such that the conversion module is not subjected to environmental effects if the valve is closed (e.g. in the passive, unused state of the measurement instrument).

The proposed instrument and method are described here using the specific example of measuring NO₂ after converting NO into NO₂; however the combination of gas converter and gas dehumidifier is claimed in general, also for other gases. In general, the respiratory gas is, in the first stage, routed through the dehumidifier. The latter is designed such that it constitutes a defined and limited flow resistance for the respiratory gas and also ensures a large contact surface of the desiccant for the respiratory gas. In this case, e.g. the use of a chamber with a loose and open-pored layering of a granulate, which contains the desiccant, or else the use of a structure with a multiplicity of channels (like a catalytic converter), where the surface of the channels have been provided with the desiccant, constitute suitable geometries.

Suitable desiccants are substances that strongly and in a well-defined fashion bind air moisture but let the target gas pass in an unimpeded fashion, e.g. substances that have a suitable internal hydrophilic pore structure such as e.g. silica gel, zeolites, hygroscopic salts and minerals such as e.g. calcium chloride, bentonite, clays, hydrophilic polymers such as e.g. dried polydextrose, polysiloxane, hydrophilic oxides such as e.g. P₂O₅, SO₃.

By way of example, an NO₂-sensitive sensor on the basis of a transistor can be used as a gas sensor 15. When using nitrogen oxide detection according to the principle of the output work measurement, various field-effect transistors are known in which the gas-sensitive layer is embodied as a gate electrode. This gate electrode can be separated from the so-called channel region of the field-effect transistor by an air gap. The basis for a detecting measurement signal is the change in the potential between gate and channel region (ΔV_{G}). By way of example, the German patent applications DE 198 14 857.7 and DE 199 56 806.5 describe hybrid flip-chip designs of gas sensors, which are embodied as CMOS transistors. A gas sensor can moreover be equipped with two field-effect transistors, the regulation behavior of which is equalized by air gaps of approximately the same size between channel region and gate electrode and the sensor layers of which can be read out separately. The German patent application DE 199 56 744.1 describes how the spacing between gate electrode and channel region of a field-effect transistor is reproducibly presentable by very precise spacers. Another embodiment provides for the gas-sensitive material to be applied to the channel region or on the gate in porous form.

Gas-sensitive layers for use in a so-called SG-FET (suspended gate field-effect transistor) can advantageously be porphin dyes such as e.g. phthalocyanines with copper or lead as central atom. In the case of sensor temperatures between 50° C. and 120°, nitrogen oxide sensitivities can be detected down to the lower ppb-range. The detection is usually targeted at nitrogen dioxide.

Other materials suitable for use in gas-sensitive field-effect transistors as gas-sensitive layers for detecting nitrogen oxide, more particularly nitrogen dioxide, are fine crystalline metal oxides operated at temperatures between 80° C. and 150° C. In particular, these can be SnO₂, WO₃, In₂O₃; salts from the carbonate system such as barium carbonate or polymers such as e.g. polysiloxane are also feasible.

The conversion module is preferably provided as close to the sensor as possible, e.g. on the inlet opening to the measurement chamber, or integrated into the measurement chamber itself so that the converted gas can be measured immediately where possible.

In one embodiment, dehumidification module and converter module are housed together in a disposable such that measurement gas flows through the dehumidification module first.

In an alternative embodiment, the disposable has a modular design, and so the dehumidification and the converter module can be replaced separately. This is advantageous if the two modules are used up at different speeds and can therefore be used in significantly differing numbers of measurement cycles.

In a further embodiment, the disposable additionally contains a particle filter in order to prevent the measuring instrument from being contaminated by bacteria. By way of example, appropriate HEPA filters (high efficiency particulate air filters) are suitable for this. The filter should have sufficiently fine pores to filter bacteria or viruses or similar contaminants from the air flow, but at the same time offer a low flow resistance.

As per a further embodiment, the conversion module with air dehumidifier is integrated into the sensor element itself (hybrid or monolithic). This can be brought about by a multi-layered design (e.g. dehumidifier layer, oxidation catalyst layer, sensor layer) or by a monolithic design (the sensor surface is on the same substrate body and has been—homogeneously or heterogeneously—mixed with the catalytically active material and dehumidification means).

Furthermore, provision can be made for a heating apparatus or a drying apparatus in order to regenerate the device for air dehumidification.

A heating apparatus can preferably be integrated onto the surface or into the material of the conversion module and regenerates the oxidative capabilities and/or the capacity of the module for air dehumidification again.

The heating apparatus can be automatically put into operation, controlled, for example, by measuring the operational hours or by measuring the gas flow through the module. In another embodiment, a calibration gas with a defined NO concentration is applied to the gas analysis instrument at selectable time intervals for quality control or calibration purposes. This calibration procedure can also serve for measuring the degree of efficiency of the conversion module and for activating the regeneration if the degree of efficiency drops.

As per one embodiment, the desiccant is designed (excess material) such that a constant moisture content of the respiratory gas is obtained thereafter, even in the case of varying moisture content of the respiratory gas (e.g. as a result of increased body temperature).

Use can also be made of a hypothetically moisture-dependent sensor.

As per one embodiment, the desiccant is applied on an open-pore matrix material for setting a defined respiratory-gas resistance. Alternatively, an open-pore material or fiber braiding may be provided elsewhere in the apparatus in order to define a respiratory-gas resistance in the instrument.

Substantial advantages of the overall system lie in that use is made of a non-invasive measurement method. The measurements can be repeated in great number and can therefore also be used in particular for monitoring progress in therapies, for diagnosing asthma in children, for early detection of asthma or for preventative medical measures. As a result of using non-depleting catalysts, the apparatus according to various embodiments does not require much servicing and allows cost-effective measurements. Hence the system presented here is also suitable for use outside clinics and medical practices.

What is claimed is:

1. An apparatus for measuring at least one gas analyte in exhaled air using a gas sensor unit with at least one gas sensor, comprising
a device for gas conversion, and
a device for air dehumidification arranged upstream of the device for gas conversion.

2. The apparatus according to claim 1, further comprising an indicator that displays the degree of water absorption or displays if the water content of the air exceeds a critical threshold after flowing through the device for air dehumidification.

3. The apparatus according to claim 1, further comprising a chemical desiccant for dehumidifying air.

4. The apparatus according to claim 3, comprising an apparatus for regenerating the chemical desiccant.

5. The apparatus according to claim 1, further having an electrical drying means for dehumidifying air.

6. The apparatus according to claim 1, wherein at least one of the device for air dehumidification and the device for gas conversion are provided as a consumable in a separate unit.

7. The apparatus according to claim 1, further comprising a particle filter.

8. The apparatus according to claim 1, further comprising a one-way valve such that a user is no longer able to suck in and re-inhale air already exhaled into the apparatus.

9. The apparatus according to claim 1, wherein the at least one gas analyte is nitrogen monoxide and the device for gas conversion is a device for oxidizing nitrogen monoxide to nitrogen dioxide.

10. The apparatus according to claim 9, wherein the gas sensor is an NO₂-sensitive FET-sensor.

11. An method for measuring at least one gas analyte in exhaled air, comprising:
dehumidifying a gas by a device for air dehumidification;
converting the dehumidified gas by a device for gas conversion; and
using a gas sensor unit with at least one gas sensor to measure gas from the device for gas conversion.

12. The method according to claim 11, displaying the degree of water absorption or displaying if the water content of the air exceeds a critical threshold after flowing through the device for air dehumidification.

13. The method according to claim 11, further providing a chemical desiccant for dehumidifying air.

14. The method according to claim 13, further comprising regenerating the chemical desiccant by an apparatus for regenerating.

15. The method according to claim 13, further comprising dehumidifying air by an electrical drying means.

16. The method according to claim 1, wherein at least one of the device for air dehumidification and the device for gas conversion are provided as a consumable in a separate unit.

17. The method according to claim 1, further comprising filtering the gas by a particle filter.

18. The method according to claim 1, further providing a one-way valve such that a user is no longer able to suck in and re-inhale air already exhaled into the apparatus.

19. The method according to claim 1, wherein the at least one gas analyte is nitrogen monoxide and the device for gas conversion is a device for oxidizing nitrogen monoxide to nitrogen dioxide.

20. The method according to claim 19, wherein the gas sensor is an NO₂-sensitive FET-sensor.
